(19) United States
(12) Patent Application Publication
(10) Pub. No.: US 2022/0249768 A1
(43) Pub. Date: Aug. 11, 2022

(54) CARTRIDGE ADAPTER FOR DRUG DELIVERY DEVICE

(71) Applicant: Becton, Dickinson and Company, Franklin Lakes, NJ (US)

(72) Inventor: Margarita Falkovich, Paramus, NJ (US)

(21) Appl. No.: 17/610,719

(22) PCT Filed: May 29, 2020
(86) PCT No.: PCT/US2020/035127

§ 371 Date: Nov. 12, 2021

Related U.S. Application Data
(60) Provisional application No. 62/854,411, filed on May 30, 2019.

(51) Int. Cl. A61M 5/145; A61M 5/142

(52) U.S. Cl. CPC A61M 5/1456; A61M 2005/14252; A61M 5/14248

(57) ABSTRACT

A cartridge adapter for a drug delivery device, the cartridge adapter includes a body having a first end and a second end positioned opposite the first end, with the body including a first clamp member, a second clamp member spaced from the first clamp member, and a connection interface configured to engage a corresponding connection interface of a drug delivery device. The first and second clamp members are moveable relative to each other and configured to receive and engage a plurality of different sized cartridges for a drug delivery device.

### CROSS-REFERENCE TO RELATED APPLICATION

The present application claims priority to U.S. Provisional Application No. 62/854,411, entitled “Cartridge Adapter for Drug Delivery Device”, filed May 30, 2019, the entire disclosure of which is hereby incorporated by reference.

### BACKGROUND OF THE INVENTION

### Field of the Invention

The present disclosure relates generally to a drug delivery device and, in particular, to a cartridge adapter for a drug delivery device.

### Description of Related Art

Various types of automatic injection or drug delivery devices have been developed to allow drug solutions and other liquid therapeutic preparations to be administered by untrained personnel or to be self-injected. Generally, these devices include a reservoir that is pre-filled with the liquid therapeutic preparation, and some type of automatic needle-injection mechanism that can be triggered by the user. When the volume of fluid or drug to be administered is generally below a certain volume, such as 1 mL, an auto-injector is typically used, which typically has an injection time of about 10 to 15 seconds. When the volume of fluid or drug to be administered is above 1 mL, the injection time generally becomes longer resulting in difficulties for the patient to maintain contact between the device and the target area of the patient's skin. Further, as the volume of drug to be administered becomes larger, increasing the time period for injection becomes desirable. The traditional method for a drug to be injected slowly into a patient is to initiate an IV and inject the drug into the patient's body slowly. Such a procedure is typically performed in a hospital or outpatient setting.

Certain devices allow for self-injection in a home setting and are capable of gradually injecting a liquid therapeutic preparation into the skin of a patient. In some cases, these devices are small enough (both in height and in overall size) to allow them to be “worn” by a patient while the liquid therapeutic preparation is being infused into the patient. These devices typically include a pump or other type of discharge mechanism to force the liquid therapeutic preparation to flow out of a reservoir and into the injection needle. Such devices also typically include a valve or flow control mechanism to cause the liquid therapeutic preparation to begin to flow at the proper time and a triggering mechanism to initiate the injection.

### SUMMARY OF THE INVENTION

In one aspect, a cartridge adapter for a drug delivery device includes a body having a first end and a second end positioned opposite the first end, with the body including a first clamp member, a second clamp member spaced from the first clamp member, and a connection interface configured to engage a corresponding connection interface of a drug delivery device. The first and second clamp members are moveable relative to each other and configured to receive and engage a plurality of different sized cartridges for a drug delivery device.

The first clamp member may extend from the first end of the body to the second end of the body, and the second clamp member may extend from the first end of the body to the second end of the body. The body and the first and second clamp members may be integrally formed with each other. The connection interface may be a dovetail. The first and second clamp members may be arcuate. The body, the first and second clamp members, and the connection interface may be integrally formed with each other.

In a further aspect, a drug delivery device includes a housing including a first connection interface, a first cartridge configured to receive a medicament, with the cartridge having a first diameter, a drive assembly received within the housing and configured to engage the cartridge and dispense medicament from the cartridge, a needle actuator assembly received within the housing, with the needle actuator assembly including a patient needle configured to pierce a patient's skin, and a cartridge adapter including a body having a first end and a second end positioned opposite the first end, with the body including a first clamp member, a second clamp member spaced from the first clamp member, and a second connection interface configured to engage the first connection interface. The first and second clamp members are moveable relative to each other, with the first and second clamp members receiving and engaging the first cartridge.

The drug delivery device may further include a second cartridge configured to receive a medicament, with the second cartridge having a second diameter. The second diameter is larger than the first diameter, and the first and second clamp members are configured to receive and engage the second cartridge. The first clamp member may extend from the first end of the body to the second end of the body, and the second clamp member may extend from the first end of the body to the second end of the body. The body and the first and second clamp members may be integrally formed with each other. The first connection interface may be a recess and the second connection interface may be a dovetail received by the recess. The first and second clamp members may be arcuate. The body, the first and second clamp members, and the connection interface may be integrally formed with each other.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a conventional drug delivery system.

FIG. 2 is a perspective, cross-sectional view of the drug delivery system of FIG. 1.

FIG. 3 is a front, cross-sectional view of the drug delivery system of FIG. 1.

FIG. 4 is a top view of the drug delivery system of FIG. 1, showing a top portion of the housing removed and the drug delivery system in a pre-use position.

FIG. 5 is a top, cross-sectional view of the drug delivery system of FIG. 1, showing the drug delivery system in a pre-use position.

FIG. 6 is a front, cross-sectional view of the drug delivery system of FIG. 1, showing the drug delivery system in a pre-use position.

FIG. 7 is a top view of the drug delivery system of FIG. 1, showing a top portion of the housing removed and the drug delivery system in an initial actuation position.

FIG. 8 is a top, cross-sectional view of the drug delivery system of FIG. 1, showing the drug delivery system in an initial actuation position.

FIG. 9 is a front, cross-sectional view of the drug delivery system of FIG. 1, showing the drug delivery system in an initial actuation position.

FIG. 10 is a top view of the drug delivery system of FIG. 1, showing a top portion of the housing removed and the drug delivery system in a use position.

FIG. 11 is a top, cross-sectional view of the drug delivery system of FIG. 1, showing the drug delivery system in a use position.

FIG. 12 is a front, cross-sectional view of the drug delivery system of FIG. 1, showing the drug delivery system in a use position.

FIG. 13 is a top view of the drug delivery system of FIG. 1, showing a top portion of the housing removed and the drug delivery system in a post-use position.

FIG. 14 is a top, cross-sectional view of the drug delivery system of FIG. 1, showing the drug delivery system in a post-use position.

FIG. 15 is a front, cross-sectional view of the drug delivery system of FIG. 1, showing the drug delivery system in a post-use position.

FIG. 15A is a front, cross-sectional view of the drug delivery system of FIG. 1, showing a pad with the drug delivery system in a pre-use position.

FIG. 15B is a perspective, cross-sectional view of the drug delivery system of FIG. 1, showing a pad with the drug delivery system in a pre-use position.

FIG. 15C is a perspective, cross-sectional view of the drug delivery system of FIG. 1, showing a pad with the drug delivery system in a pre-use position.

FIG. 16 is a partial cross-sectional view of the drug delivery system of FIG. 1, showing a valve assembly.

FIG. 17 is an exploded perspective view of a cartridge adapter for a drug delivery device according to one aspect of the present invention.

FIG. 18 is a perspective view of the cartridge adapter of FIG. 17 according to one aspect of the present invention, showing the cartridge adapter connected with a drug delivery device with a cartridge received within the cartridge adapter.

FIG. 19 is a perspective view of the cartridge adapter of FIG. 17 according to one aspect of the present invention.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

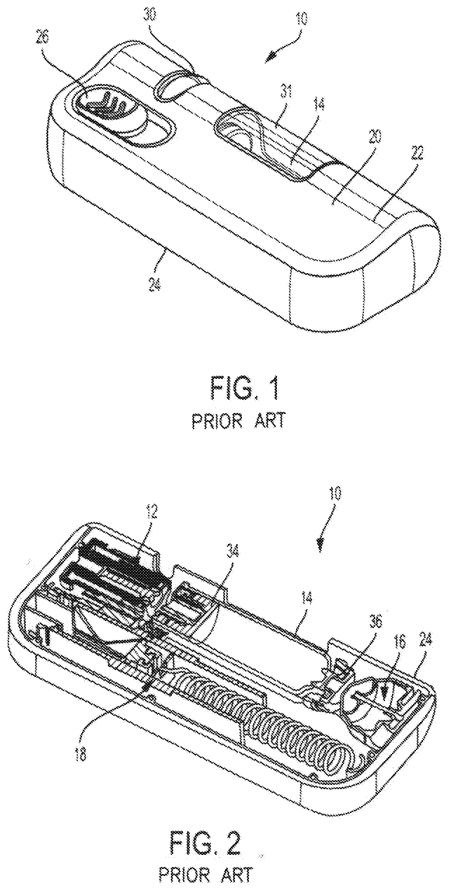

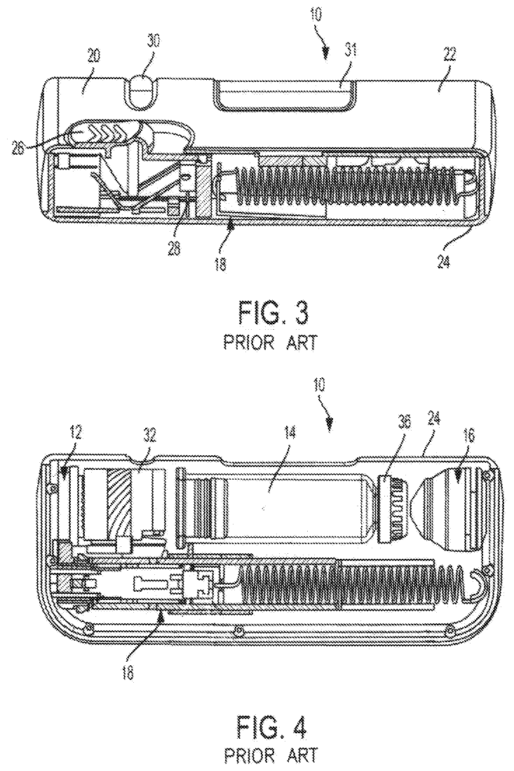

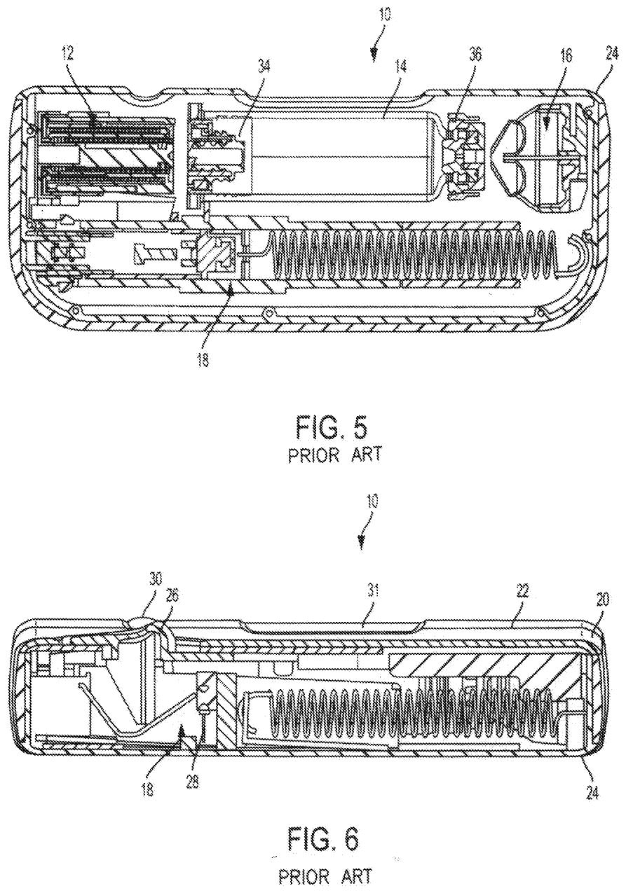

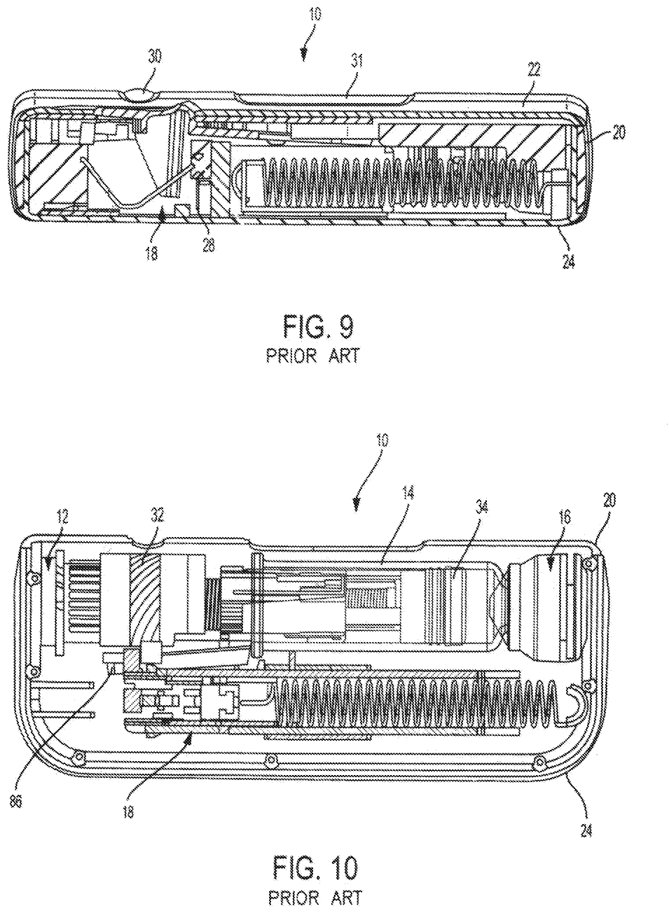

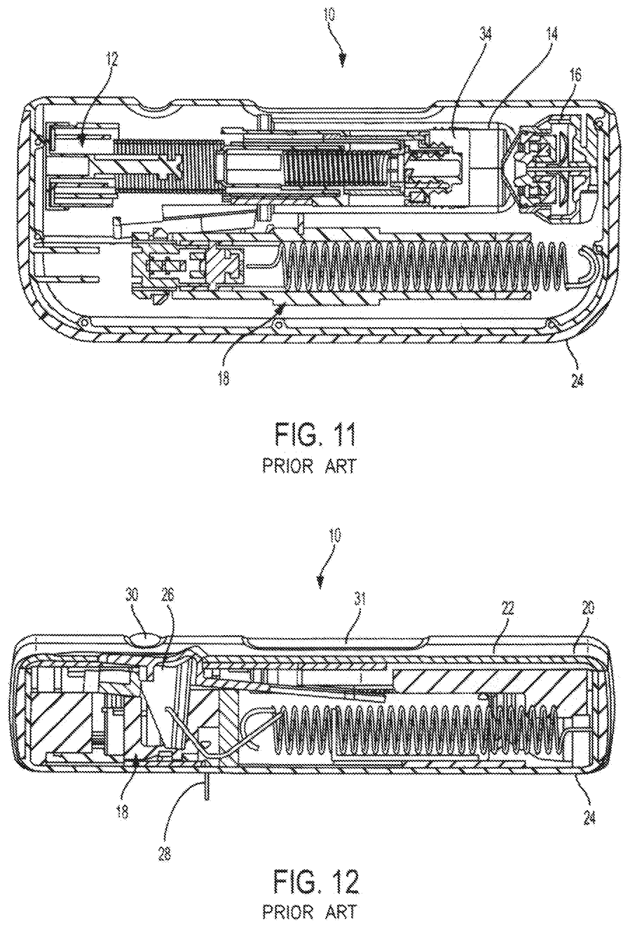

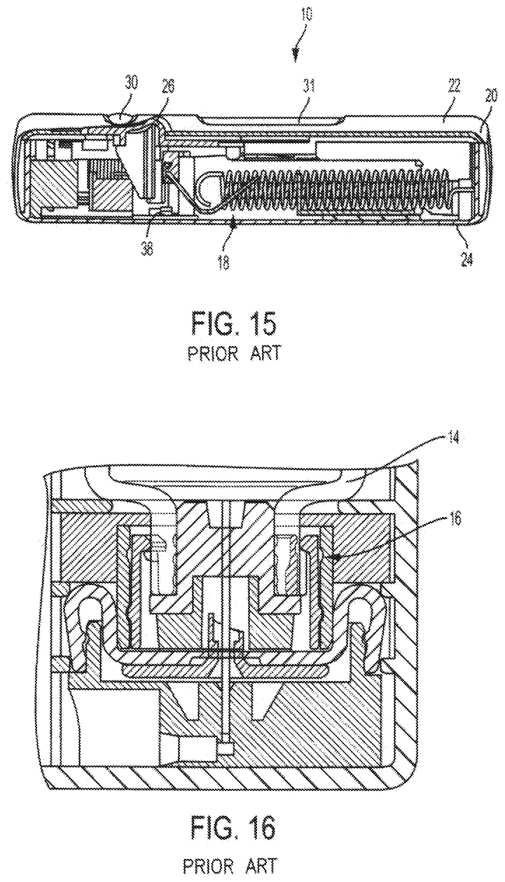

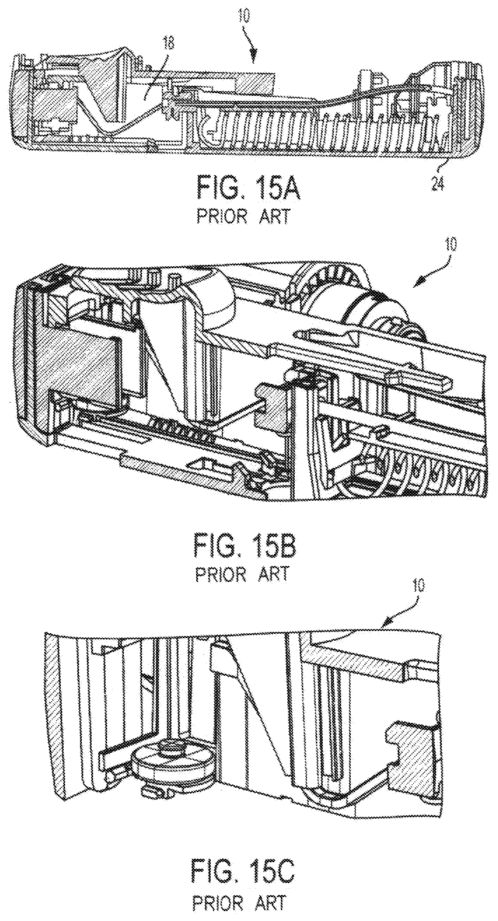

Referring to FIGS. 1-16, a conventional drug delivery system 10 includes a drive assembly 12, a container 14, a valve assembly 16, and a needle actuator assembly 18. The drive assembly 12, the container 14, the valve assembly 16, and the needle actuator assembly 18 are at least partially positioned within a housing 20. The housing 20 includes a top portion 22 and a bottom portion 24, although other suitable arrangements for the housing 20 may be utilized. In one aspect, the drug delivery system 10 is an injector device configured to be worn or secured to a user and to deliver a predetermined dose of a medicament provided within the container 14 via injection into the user. The system 10 may be utilized to deliver a “bolus injection” where a medicament is delivered within a set time period. The medicament may be delivered over a time period of up to 45 minutes, although other suitable injection amounts and durations may be utilized. A bolus administration or delivery can be carried out with rate controlling or have no specific rate controlling. The system 10 may deliver the medicament at a fixed pressure to the user with the rate being variable. The general operation of the system 10 is described below in reference to FIGS. 1-16.

Referring again to FIGS. 1-16, the system 10 is configured to operate through the engagement of an actuation button 26 by a user, which results in a needle 28 of the needle assembly 18 piercing the skin of a user, the actuation of the drive assembly 12 to place the needle 28 in fluid communication with the container 14 and to expel fluid or medicament from the container 14, and the withdrawal of the needle 28 after injection of the medicament is complete. The general operation of a drug delivery system is shown and described in International Publication Nos. 2013/155153 and 2014/179774, which are hereby incorporated by reference in their entirety. The operation of the system 10 is also shown and described in U.S. Publication No. 2017/0354788, which is hereby incorporated by reference in its entirety. The housing 20 of the system 10 includes an indicator window 30 for viewing an indicator arrangement 32 configured to provide an indication to a user on the status of the system 10 and a container window 31 for viewing the container 14. The indicator window 30 may be a magnifying lens for providing a clear view of the indicator arrangement 32. The indicator arrangement 32 moves along with the needle actuator assembly 18 during use of the system 10 to indicate a pre-use status, use status, and post-use status of the system 10. The indicator arrangement 32 provides visual indicia regarding the status, although other suitable indicia, such an auditory or tactile, may be provided as an alternative or additional indicia.

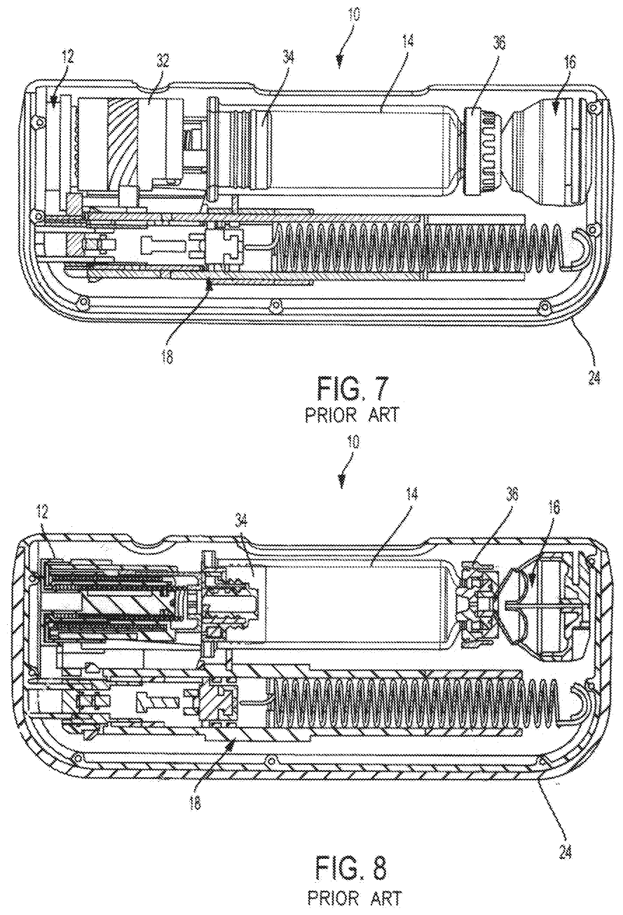

Referring to FIGS. 4-6, during a pre-use position of the system 10, the container 14 is spaced from the drive assembly 12 and the valve assembly 16 and the needle 28 is in a retracted position. During the initial actuation of the system 10, as shown in FIGS. 7-9, the drive assembly 12 engages the container 14 to move the container 14 toward the valve assembly 16, which is configured to pierce a closure 36 of the container 14 and place the medicament within the container 14 in fluid communication with the needle 28 via a tube (not shown) or other suitable arrangement. The drive assembly 12 is configured to engage a stopper 34 of the container 14, which will initially move the entire container 14 into engagement with the valve assembly 16 due to the incompressibility of the fluid or medicament within the container 14. The initial actuation of the system 10 is caused by engagement of the actuation button 26 by a user, which releases the needle actuator assembly 18 and the drive assembly 12 as discussed below in more detail. During the initial actuation, the needle 28 is still in the retracted position and about to move to the extended position to inject the user of the system 10.

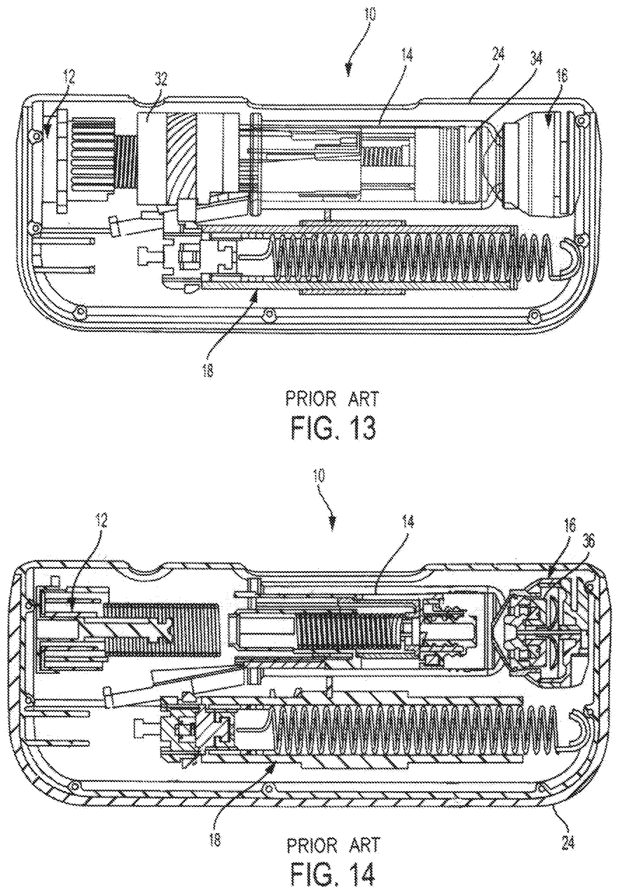

During the use position of the system 10, as shown in FIGS. 10-12, the needle 28 is in the extended position at least partially outside of the housing 20 with the drive assembly 12 moving the stopper 34 within the container 14 to deliver the medicament from the container 14, through the needle 28, and to the user. In the use position, the valve assembly 16 has already pierced a closure 36 of the container 14 to place the container 14 in fluid communication with the needle 28, which also allows the drive assembly 12 to move the stopper 34 relative to the container 14 since fluid is able to be dispensed from the container 14. At the post-use position of the system 10, shown in FIGS. 13-15, the needle 28 is in the retracted position and engaged with a pad 38 to seal the needle 28 and prevent any residual flow of fluid or medicament from the container 14.

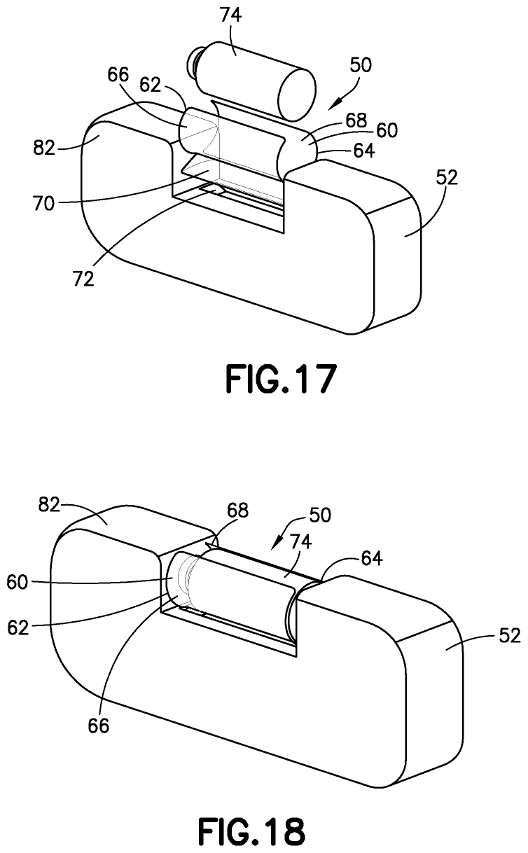

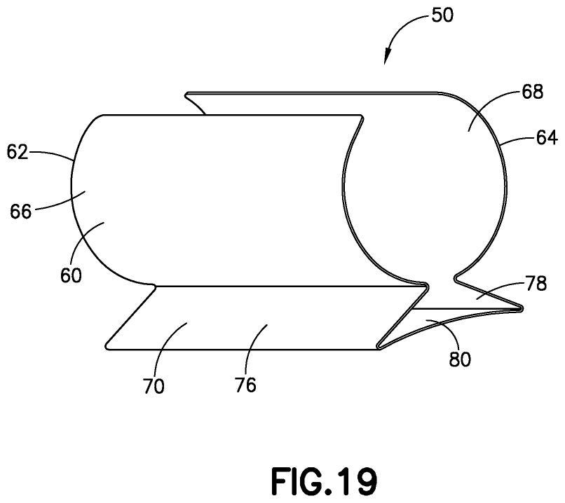

Referring to FIGS. 17-19, a cartridge adapter 50 for a drug delivery device 52 is shown. The drug delivery device 52 may be the same as the system 10 shown in FIGS. 1-16 and described above. The cartridge adapter 50 may be integrated in the system 10 with the system 10 operating in the same manner as discussed above. The cartridge adapter 50 includes a body 60 having a first end 62 and a second end 64 positioned opposite the first end 62. The body 60 includes a first clamp member 66 and a second clamp member 68 spaced from the first clamp member 66. A connection interface 70 is configured to engage a corresponding connection 72 interface of the drug delivery device 52. The first and second clamp members 66, 68 are moveable relative to each other and configured to receive and engage a plurality of different sized cartridges 74 for the drug delivery device 52. The cartridge 74 may be the same as the container 14 discussed above and shown in FIGS. 1-16. The cartridge adapter 50 is configured to receive cartridges 74 having different sized diameters and different lengths.

As shown in FIGS. 17 and 18, the cartridge 74 may be inserted into the cartridge adapter 50 by engaging the first and second clamp members 66, 68 with the cartridge 74, which causes the first and second clamp members 66, 68 to move further away from each other thereby creating a biasing force to engage and secure the cartridge 74 between the first and second clamp members 66, 68. The first and second clamp members 66, 68 are arcuate such that the shape of the first and second clamp members 66, 68 generally correspond to the outer surface of the cartridge 74, although other suitable shapes and configurations may be utilized.

The first clamp member 66 extends from the first end 62 of the body 60 to the second end 64 of the body 60. The second clamp member 68 also extends from the first end 62 of the body 62 to the second end 64 of the body 60. The connection interface 70 is a dovetail that is received by and engaged with the corresponding connection 72 interface, which is formed by a dovetail recess defined by a housing 82 of the drug delivery device 52, although other suitable connection interfaces may be utilized. The connection interface 70 includes a first flange 76 extending from the first clamp member 66 and a second flange 78 extending from the second clamp member 68 with a connection portion 80 extending between the first and second flanges 76, 78. The first and second flanges 76, 78 may be planar and the connection portion 80 may be arched. The connection interface 70 may be flexible to facilitate the connection of the connection interface 70 of the cartridge adapter 50 with the corresponding interface 72 of the drug delivery device 52. The cartridge adapter 50 allows the cartridge 74 to be installed onto the drug delivery device 52 from outside of the housing 82. The body 60, the first and second clamp members 66, 68, and the connection interface 70 are integrally formed with each other, although other suitable arrangements may be utilized. In particular, the first and second clamp members 66, 68, and the connection interface 70 are formed from a single sheet of material. The body 60 may be formed from a polymeric material, although other suitable materials or combination of materials may be utilized.

Elements of one disclosed aspect can be combined with elements of one or more other disclosed aspects to form different combinations, all of which are considered to be within the scope of the present invention.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A cartridge adapter for a drug delivery device, the cartridge adapter comprising:
a body having a first end and a second end positioned opposite the first end, the body comprising a first clamp member, a second clamp member spaced from the first clamp member, and a connection interface configured to engage a corresponding connection interface of a drug delivery device, the first and second clamp members are moveable relative to each other and configured to receive and engage a plurality of different sized cartridges for a drug delivery device.

2. The cartridge adapter of claim 1, wherein the first clamp member extends from the first end of the body to the second end of the body, and wherein the second clamp member extends from the first end of the body to the second end of the body.

3. The cartridge adapter of claim 1, wherein the body and the first and second clamp members are integrally formed with each other.

4. The cartridge adapter of claim 1, wherein the connection interface comprises a dovetail.

5. The cartridge adapter of claim 1, wherein the first and second clamp members are arcuate.

6. The cartridge adapter of claim 1, wherein the body, the first and second clamp members, and the connection interface are integrally formed with each other.

7. A drug delivery device comprising:
a housing including a first connection interface;
a first cartridge configured to receive a medicament, the cartridge having a first diameter;
a drive assembly received within the housing and configured to engage the cartridge and dispense medicament from the cartridge;
a needle actuator assembly received within the housing, the needle actuator assembly comprising a patient needle configured to pierce a patient's skin; and
a cartridge adapter comprising a body having a first end and a second end positioned opposite the first end, the body comprising a first clamp member, a second clamp member spaced from the first clamp member, and a second connection interface configured to engage the first connection interface, the first and second clamp members are moveable relative to each other, the first and second clamp members receiving and engaging the first cartridge.

8. The drug delivery device of claim 7, further comprising a second cartridge configured to receive a medicament, the second cartridge having a second diameter, the second diameter is larger than the first diameter, the first and second clamp members are configured to receive and engage the second cartridge.

9. The drug delivery device of claim 7, wherein the first clamp member extends from the first end of the body to the second end of the body, and wherein the second clamp member extends from the first end of the body to the second end of the body.

10. The drug delivery device of claim 7, wherein the body and the first and second clamp members are integrally formed with each other.

11. The drug delivery device of claim 7, wherein the first connection interface comprises a recess and the second connection interface comprises a dovetail received by the recess.

12. The drug delivery device of claim 7, wherein the first and second clamp members are arcuate.

13. The drug delivery device of claim 7, wherein the body, the first and second clamp members, and the second connection interface are integrally formed with each other.
